(19) United States
(12) Patent Application Publication
(10) Pub. No.: US 2014/0377201 A1
(43) Pub. Date: Dec. 25, 2014

(54) SKIN-WHITENING COMPOSITION CONTAINING EXTRACTS FROM TREES INCLUDING PAPER MULBERRY

(71) Applicant: Amorepacific Corporation, Seoul (KR)

(72) Inventors: Jin Young LEE, Yongin-si (KR); Hyung Jun Jun LIM, Yongin-si (KR)

(73) Assignee: AMOREPACIFIC CORPORATION, Seoul (KR)

(21) Appl. No.: 14/478,195

(22) Filed: Sep. 5, 2014

Related U.S. Application Data
(62) Division of application No. 13/807,869, filed on Dec. 31, 2012.

(30) Foreign Application Priority Data
Jul. 2, 2010 (KR) 10-2010-0064031

(51) Int. Cl. A61K 8/97; A61Q 19/02

(52) U.S. Cl. CPC A61K 8/97; A61Q 19/02; A61K 2800/782
USPC 424/62

(57) ABSTRACT

Disclosed is a skin-whitening composition comprising a paper mulberry extract, and more particularly to a skin-whitening composition containing an extract of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold, which can be used safely on the skin without causing side effects and has excellent effects of inhibiting melanin production and pigmentation.

The present application is a divisional of U.S. application Ser. No. 13/807,869 filed Dec. 31, 2012, which is a National Stage of International Application No. PCT/KR 2011/004889, filed on Jul. 4, 2011, which claims the benefit of priority from Korean Patent Application No. KR 10-2010-0064031, filed on Jul. 2, 2010, the contents of which are herein incorporated by reference in their entirety.

### TECHNICAL FIELD

The present invention relates to a skin-whitening composition containing a paper mulberry extract, and more particularly to a skin-whitening composition containing an extract of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold, which can be used safely on the skin without causing side effects and has excellent effects of inhibiting melanin production and pigmentation.

### BACKGROUND ART

Human skin color is determined. by various actors, including the activity of melanocytes which. produce the melanin pigment, distribution of blood vessels, skin thickness, the presence or absence of pigments such as carotenods and bilirubin, or the like. Of the factors, the dark. pigment melanin which is produced from melanocytes by the action of various enzymes including tyrosinase is the most important.

The production of melanin is affected genetic factors, physiological factors such as hormone secretion, stress, etc. and. environmental factors such as UV radiation. Melanin present in the skin plays an important role in protecting the skin. from UV, etc. However, it is known that excessive production of melanin plays major roles in accelerating pigmentation and skin aging and inducing skin cancer.

From long ago, ascorbic acid, kojic acid, arbutin, hydroquinone, glutathione, their derivatives, or compounds having tyrosinase inhibitory activity have been used in cosmetics or drugs in order to treat or ameliorate anomalous skin pigmentation and excessive melanin pigmentation caused by exposure to UV. However, their use is restricted because of insufficient skin. whitening effect, safety issue on the skin, formulation instability when used in cosmetics, or the like.

Many studies have been conducted to fine raw materials which have excellent skin whitening effects while overcoming the limitations of conventional skin whitening substances. For example, Korean Patent Registration. No. 1993-0010548 discloses the skin whitening effects of Broussonetia kazinoki Sieb or Broussonetia papyrifera extracts, and Korean Patent Laid-Open Publication No. 2006-0121496 discloses the skin whitening effects of Broussonetia kazinoki var. humilis extracts. However, prior to the present invention, there has been no report of the skin whitening effects of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold.

### DISCLOSURE

### Technical Problem

Accordingly, the present inventors have conducted studies on natural materials having skin whitening effects and, as a result, have found that extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold have excellent skin whitening effects compared to extracts of Broussonetia kazinoki Sieb, Broussonetia papyrifera and Broussonetia kazinoki var. humilis extracts, thereby completing the present invention.

Therefore, it is an object of the present invention provides a skin-whitening composition which exhibits excellent skin whitening effects and, at the same time, can be used safely on the skin without causing side effects.

### Technical Solution

In order to accomplish the above objects, the present invention provides a skin-whitening composition containing, as an active ingredient, one or more selected from the group consisting of extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold.

### Advantageous Effects

A skin-whitening composition according to the present invention has not only excellent effects on the inhibition of tyrosinase activity and melanin production, but also excellent skin safety.

### BEST MODE

The present invention relates to a skin-whitening composition containing, as an active ingredient, an extract of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold, which can be used safely on the skin without causing side effects and has excellent effects of inhibiting melanin production and pigmentation.

Acer barbinerve Max. that is used in the present invention has leaves, which are opposite each other, broad ovate or nearly circular, acuminate, cordate or truncate at the base, have a length of 5-10 cm and a width of 4-7 cm, are divided into 5 parts at the edge, hairy on the surface, and have hairs on the back surface in the case of young leaves, in which the hairs on the back surface disappear gradually to remain only at the veins. The leaves are doubly serrated at the edge and are not serrated at the end of the lobe, and the petiole is 4-13 cm in length and has fine hairs. The flowers are dioecious and bloom in June, 4-7 flowers form a raceme, and the peduncle is hairy. The female flower sprouts from the shoot apex, and the flower stalk is 1-2 cm in length. The male flower sprouts from the upper part of last season's branches and has 4 petals which are oval and yellowish green. The fruit is a samara, dehisces at an obtuse or right angle, have a length of 3-3.5 cm and a width of 8-12 mm, comprises many wrinkles, and ripens in October. The wings are lanceolate, and the small fruit stalk is 1.5-2 cm in length. 5-7 fruits cluster, and the nutlets are spherical. The bark. is gray hr own in color and is smooth, and the twig is yellowish, but sometimes reddish, and has hairs.

Acer barbinervis var. glabrescens that is used in the present invention grows in mountainous districts to a height of 3-10 m. The bark is smooth and is grayish brown in color, and the twig is yellowish or reddish. The leaves are opposite each other, are broad oval or round in shape, and 5-10 cm in length. The leaves are divided into five lobes, like the hand palm, and have little or no hair on the surface. The lobes are pointed, doubly serrated, and serrated at the end. The petiole is long and has fine hairs. The flowers are monoecious and open in May through June, and 4-7 flowers a raceme. The edge of the receptacle is ring-shaped. The male flower has 4 stamens and 2 stigmas. The fruit is a samara, has lancet based wings, ripens in October and dehisces at an obtuse or right. angle. The plant. is a Korean endemic plant that is distributed in Gangwon-do, Pyeonganbuk-do and Jiri Mountain.

Wikstroemia trichotoma that is used in the present invention is a deciduous shrub of the family Thymelaceae of the order Myrtales and grows in mountainous districts to a height of about 1 m. The twig is hairless and reddish brown in color. The leaves are opposite each other, oval in shape, 3-4 cm in length and 1-3 cm in width. The leaves are blunt at the end, blunt or pointed at the base, hairy at both ends, and plain at the edge. The back side of the leaves is white, and the petiole has a length of about 2 mm. The flowers are bisexual, bloom in July through August and are yellow in color, and 7-15 flowers form a raceme. The calyx is 708 mm in length, and its end is divided into 4 parts and spreads laterally. The flower has 8 stamens in 2 rows and 1 stigma. The ovary is oval and has a stalk. The fruit is a dry fruit is long-oval in shape and has a length of about 5 mm. It is narrow at both ends, hairless and ripens in September through October. The bark is used to make Korean paper or ropes. The plant is distributed in Korea (Jeollanam-do, Gyeongsangnam-do and Incheon-si) and Japan. In the case of W. sikokiana, the leaves are alternate, and the flowers are clustered at the end of the branch. The bark of W. sikokiana is used to make paper and grows in the middle part of Japan.

Acer tschonoskii var. rubripes that is used in the present invention is also known as Dan Pung Rae in Korean and grows in deep mountains to a height of about 10 m. The bark is ash in color, but the twig is purple. The leaves are opposite each other, oval in shape, and 5-9 cm in length. The leaves are pointed at the end and heart-shaped at the base and have dense hairs at the veins of the back side. The leaves are divided into 3-5 lobes at the edge and are serrated at the edge of the lobe, and the petiole is 2-5 cm in length and reddish. The flowers are monoecious, bloom yellow in June, and 5-10 flowers form a raceme. The raceme is 6-8 cm in length. The flower has 5 petals and 5 sepals. The fruit is a samara and ripens in October, and the wings are lancet-shaped and 5 mm in width. The plant is used as a garden tree, and the wood thereof is used for industrial applications. It is distributed in Korea, Japan, China and the Like.

Edgeworthia chrysantha that is used in the present invention is a deciduous broad a shrub and grows L3 a height of about 2 m. The branch is divided into three parts. The leaves are about 10 cm in length, lanceolate, alternate and thin. The flowers are yellow and run downward from the axilla to form a ball-shaped capitulum before the leaves are out. Each flower is about 1 cm long, cylindrical in length, divided into 4 part at the end, yellow on the inner side and has dense hairs on the outer side. It has 8 stamens, 4 of which are attached to the calyx tube so that the anther extends outward. The plant is native to China, and grows mainly in warm places, and in Korea, it is distributed in Jeju-do and the like. The bark has been used as raw material for making paper.

Broussonetia papyrifera for. oppositifolia that is used in the present invention is a deciduous broad-leaf tree of Moraceae of the order Order Urticales. It grows in sunny mountainous areas at 100-700 m above the sea. It grows to a height of about 12 m. The bark is dark-gray in color, and the twig has dense hairs. The leaves are opposite each other, and. broad oval or round in shape. The leaves are 7-20 cm in length, and the end thereof is hallowed deep, like a tail. The leaves are hairy and rough on both sides and serrated at the edge. The petiole is 3-10 cm in length and has hairs. The stipple is purple in color, oval and early sheds. The flowers are unisexual and bloom greenish brown in May through June, and the male flower spike is cylindrical in shape and runs downward from the axilla. The female flower spike is round in shape. The male flower perianth is divided into 4 parts and has 4 stamens. The female flower perianth is tubular in shape and is divided into 3-4 parts. The fruit is a round stone fruit and ripens in September. The epicarp is red, and the endocarp is hard and brown in color. Young leaves and fruits are eatable, and the bark is used to make paper. In Chinese medicine, the bark and the fruit are used as drugs for tonic, diuretic, anti-paralytic and cough. remedy applications. The plant is propagated by seeding, planting a cutting or division. It is a kind of Broussonetia papyrifera and is characterized in that the leaves are opposite each other. It is distributed in Korea (the whole), japan, China and the like.

Broussonetia papyrifera for. lucida that is used in the present invention is a deciduous broad-leaf tree of Moraceae of the order Urticales. It grows in sunny mountainous areas at 100-700 m above the sea. It grows to a. height, of about. 12m. The bark is dark-gray in color, and the twig has dense hairs. The leaves are broad oval or round in shape and opposite each. other. The leaves are 7-20 cm in length, deep-green in color, hairless and glossy. The petiole is 3-10 cm in length and has hairs. The stipple is purple in color, oval and early sheds. The flowers are unisexual and bloom greenish brown in May through June together with the leaves, and the male flower spike is cylindrical, in shape and runs downward from the axilla of fresh branches. The male flower perianth is divided into 4 parts and has 4 stamens. The female flower spike is round in shape. The female flower perianth is divided into 3-4 parts, and the style is long like yarn. The fruit is a stone fruit. The epicarp is red, and the endocarp is hard. and brown in color and ripens in September. Young leaves and fruits are eatable, and the bark is used to make paper. In Chinese medicine, the bark and the fruit, are used as drugs for tonic, diuretic, anti-paralytic and cough remedy applications. plant propagated by seeding, planting a cutting or division. It is a kind of Broussonetia papyrifera and is characterized in that the leaves are deep-green in color, hairless and glossy. It is distributed in Korea (the whole), Japan, China and the like.

Daphne kamtschtica that is used in the present invention is a deciduous shrub of the family Thymelaceae of the order Myrtales and grows in deep mountains. It is 30-40 cm in height and, the branches are hairless. The leaves are inverted egg or lancet-shaped, pointed or blunt at the end, and 4.0-8.5 cm in length. The front side of the leaves is blue-green in color, and the back side is slightly white is are not serrate, and the petiole is 5-7 mm in length. The raceme develops from the axil of last season's branches and has 2-5 yellow flowers at the upper portion and 2-5 green flowers at the lower portion. The flowers are dioecious, and the female flowers are smaller. The calyx is yellow in color, oval or lancet-shaped, and pointed at the end. The flower has 8 stamens, and the fruit is a succulent fruit, is spherical, in shape and ripens red in the fall season. The bark is used to make paper and ropes. The plant distributed in Korea (Jeollanam-do, Gangwon-do, Pyeonganbuk-do and Hamgyeongbuk-do), Sahalin, Amur, Wūsūl\check{i} and the like.

Broussonetia kazinoki Siebold that is used in the present invention is a deciduous broad-leaf tree of Moraceae and is 2-5 m in height. The bark is yellowish brown in color. This plant is distributed. in Southeastern. Asia. and cultivated in most areas of Korea.

The extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold, which are used in the present invention, include an extract obtained by extracting the stem, root, leaf, flower or fruit of each of the plant, a concentrate obtained by concentrating some or all of the extract, and an infusion, decoction, tincture and fluid extract obtained after drying the concentrate, as well as active ingredients contained in the plants, and also the plants themselves.

The extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold according to the present invention can be easily prepared by those skilled in the art according to any method known in the art. For example, the stem, root, leaf, flower or fruit of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold is dried by any method such as natural drying or forced drying and cut finely, after which it is extracted by any method such as cold maceration, percolation or warm maceration using a polar solvent such as water, ethanol, butanol or acetone, or a non-polar solvent such as ether, hexane, benzene, chloroform or ethyl acetate, or a mixed solvent of the non-polar solvent and the polar solvent, or a solvent such as alkaline water, or vegetable oil such as bean oil or sesame oil, thereby obtaining an extract containing an active ingredient. With the extraction process, cold maceration and percolation are preferably carried out for 12-96 hours, and warm maceration is preferably carried out at a temperature close to the reflux temperature of solvent for 0.5-24 hours depending on the kind of solvent used and the temperature of maceration. Particularly, a tincture, extract or id extract obtained by extraction. in hydrated. alcohol is preferably used.

The skin whiten composition according to the present invention. contains the extract of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold in an amount of 0.0001-90 wt %, and more preferably 0.0001-50 wt %, based on the total weight of the composition. If the content of extract in the composition is less than 0.0001 wt %, the desired skin-whitening effect cannot be obtained, and if the content is more than 90 wt %, there will be a problem in terms of skin safety or formulation.

The skin-whitening composition according to the present invention can be applied to cosmetics, drugs, foods and the like and can be in the form of oral formulations (internal use) or parenteral formulations (external use).

In addition, the skin whitening composition according to the present invention may contain, in addition to the extract of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold, other whitening ingredients capable of increasing the skin whitening effect within the range that does not impair the object of the present invention. Such other skin-whitening ingredients include arbutin, ascorbic acid derivatives and the like.

The composition according to the present invention contains a cosmetically and dermatologically acceptable medium and/or base. The composition may be formulated as a preparation for topical application. Examples of formulations for local application include solution, gel, solid or dough anhydride, emulsion prepared by dispersing oil phase in a water phase, suspension, microemulsion, microcapsule, microgranule, ionic (liposome) and/or non-ionic vesicle, cream, skin, lotion powder, spray, and conceal stick. Also, the composition for skin external application according to the present invention can be formulated as a foam composition or an aerosol composition further containing a compressed propellant. In addition, the composition according to the present invention can be prepared formulated according to a conventional method known in the art.

The skin whitening composition according to the present invention may contain additives which are conventionally field in the cosmetic field or the skin science field, for example, fatty substance, organic solvent, resolvent, thickener, gelling agent, softener, antioxidant, suspending agent, stabilizer, foaming agent, fragrance ingredient, surfactant, water, ionic or non-ionic emulsifying agent, filler, sequestering agent, chelating agent, preservative, vitamins, blocker, moisturizing agent, essential oil, dye, pigment, hydrophilic or hydrophobic activator, lipid vesicle or other components. These additives are contained in amounts which are generally used in the cosmetic field or the skin science field.

Hereinafter, the present invention will be described in further detail with reference to examples and test examples. It is to be understood, however, that these examples are for illustrative purposes only and are not to be construed to limit the scope of the present invention. Also, those skilled in the art will appreciate that various modifications, additions and substitutions are possible, without departing from the scope and spirit of the present invention as disclosed in the accompanying claims.

### EXAMPLES 1 to 9

1 kg of the dried whole plant of each of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica and Broussonetia kazinoki Siebold was added to 10 L of purified water and heated until boiling, after which it was additionally heated for 10 minutes. Then, the water was removed, and the residue was washed and then washed by addition of 10 L of purified water. The residue was air-dried, added to 20 L of 70% ethanol, connected to a reflux system, heated, and extracted under reflux for 24 hours. The extract was filtered through a 80-mesh sieve to remove the solid, and the remaining filtrate was filtered again and concentrated using a vacuum evaporator to remove the solvent, thereby obtaining solid powder of each extract.

### COMPARATIVE EXAMPLES 1 to 3

Each of Broussonetia kazinoki Sieb, Broussonetia papyrifera (L.) Vent and Broussonetia kazinoki var. humilis was extracted in the same manner as Examples 1 to 9, thereby obtaining a solid extract of each plant.

### TEST EXAMPLE 1

### Tyrosinase Inhibitory Effect

The enzyme tyrosinase used in this Test Example was a tyrosinase (SIGMA) extracted from mushrooms. First, the substrate tyrosine was dissolved in distilled water at a concentration of 0.3 mg/ml, and 1.0 ml of the solution was placed in each test tube. Then, 1.0 ml of potassium-phosphate buffer (0.1 M, pH 6.8) and 0.7 ml of distilled water were added thereto, thereby preparing a mixed solution.

Each of the extracts of Examples 1 to 9 and Comparative Examples 1 to 3 was dissolved in ethanol at a suitable concentration, and 0.2 ml (10 μg/ml) of each of the extract sample solutions was added to the mixed solution, and then allowed to react in an incubator at 37° C. for 10 minutes. As a negative control, 0.2 ml of a solvent alone was used instead of each extract sample, and as a positive control, 40 μg/ml of kojic acid (KA) was used. 0.1 ml of a tyrosinase solution (2500 unit/ml) was added to each of the reaction solutions and allowed to react in an incubator at 37° C. for 10 minutes. Then, the test tube containing the reaction solution was quenched in ice water to stop the reaction, and the absorbance at 475 nm was measured with a photoelectric spectrophotometer. Then, the tyrosinase inhibitory activity of each of the extracts was calculated using the following equation 1, and the results of the calculation are shown in Table 1 below.

Tyrosinase inhibition (%)=100−(absorbance of each extract/absorbance of control×100 [Equation 1]

TABLE 1

| Test material | Tyrosinase inhibition (%) |
|---|---|
| Control (to which no test | 0 |
| material was added) | |
| Kojic acid | 51 |
| Example 1 | 71 |
| Example 2 | 83 |
| Example 3 | 76 |
| Example 4 | 69 |
| Example 5 | 57 |
| Example 6 | 69 |
| Example 7 | 71 |
| Example 8 | 68 |
| Example 9 | 52 |
| Comparative Example 1 | 65 |
| Comparative Example 2 | 34 |
| Comparative Example 3 | 31 |

As can be seen from the results in Table 1 above, the extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold according to the present invention showed very high tyrosinase inhibitory activities, suggesting that the extracts have excellent skin whitening effects.

### TEST EXAMPLE 2

### Evaluation of Melanin Production Inhibitory Activity Using B16/F10 Melanoma Cells

Samples, containing 0.001 wt % of each of the extracts of Examples 1 to 9 and Comparative Examples 1 to 3, and 50 /μg/ml of arbutin, were used as test materials. Each of the test materials was added to a culture of B16/F10 melanoma cells (Korean Cell Line Bank) at a given concentration and incubated for 3 days. The medium as removed, and the cells were washed with PBS and lysed with 1N NaOH, and the absorbance at 405 nm was measured. As a control, cells to which no test material was added were used. The skin whitening effects of the test materials were measured by measuring the melanin production inhibitory activity of each test material in comparison with the content of melanin in the control. Melanin production inhibition (%) was calculated using the following equation 2, and the calculation results are shown in Table 2 below.

Melanin production inhibition (%)=100−(absorbance of each test material/absorbance of control)×100 [Equation 2]

TABLE 2

| Test material | Melanin production inhibition (%) |
|---|---|
| Control (to which no test | 0 |
| material was added) | |
| Arbutin | 48 |
| Example 1 | 38 |
| Example 2 | 42 |
| Example 3 | 46 |
| Example 4 | 53 |
| Example 5 | 47 |
| Example 6 | 41 |
| Example 7 | 39 |
| Example 8 | 36 |
| Example 9 | 42 |
| Comparative Example 1 | 42 |
| Comparative Example 2 | 15 |
| Comparative Example 3 | 5 |

As can be seen from the results in Table 1 above, the extracts of Acer barbinerve Max., Acer barbinervis var. glabrescens, Wikstroemia trichotoma, Acer tschonoskii var. rubripes, Edgeworthia chrysantha, Broussonetia papyrifera for. Oppositifolia, Broussonetia papyrifera for. Lucida, Daphne kamtschtica or Broussonetia kazinoki Siebold showed high melanin production inhibitory activities, suggesting that the extracts have excellent skin whitening effects.

### FORMULATION EXAMPLES 1 to 9

Milk lotions of Formulation Examples 1 to 9 were prepared using the compositions shown in Tables 3 and 4 below according to a conventional method (unit: wt %).

TABLE 3

| | Form. | Form. | Form. | Form. | Form. |
|---|---|---|---|---|---|
| | Exam- | Exam- | Exam- | Exam- | Exam- |
| Component | ple 1 | ple 2 | ple 3 | ple 4 | ple 5 |
| Extract of Example 1 | 0.1 | — | — | — | — |
| Extract of Example 2 | — | 0.1 | — | — | — |
| Extract of Example 3 | — | — | 0.1 | — | — |
| Extract of Example 4 | — | — | — | 0.1 | — |
| Extract of Example 5 | — | — | — | — | 0.1 |
| Squalane | 5.0 | 5.0 | 5.0 | 5.0 | 5.0 |
| Beeswax | 4.0 | 4.0 | 4.0 | 4.0 | 4.0 |
| Polysorbate 60 | 1.5 | 1.5 | 1.5 | 1.5 | 1.5 |
| Sorbitan sesquioleate | 1.5 | 1.5 | 1.5 | 1.5 | 1.5 |
| Liquid paraffin | 0.5 | 0.5 | 0.5 | 0.5 | 0.5 |
| Caprylic/capric | 5.0 | 5.0 | 5.0 | 5.0 | 5.0 |
| triglyceride | | | | | |
| Glycerin | 3.0 | 3.0 | 3.0 | 3.0 | 3.0 |
| Butylene glycol | 3.0 | 3.0 | 3.0 | 3.0 | 3.0 |
| Propylene glycol | 3.0 | 3.0 | 3.0 | 3.0 | 3.0 |
| Carboxyvinyl polymer | 0.1 | 0.1 | 0.1 | 0.1 | 0.1 |
| Triethanolamine | 0.2 | 0.2 | 0.2 | 0.2 | 0.2 |
| Preservative, pigment, | q.s. | q.s. | q.s. | q.s. | q.s. |
| fragrance | | | | | |
| Purified water | Balance | Balance | Balance | Balance | Balance |

TABLE 4

| | | | | | Comp. |
|---|---|---|---|---|---|
| | Form. | Form. | Form. | Form. | Form. |
| | Exam- | Exam- | Exam- | Exam- | Exam- |
| Component | ple 6 | ple 7 | ple 8 | ple 9 | ple 1 |
| Extract of Example 6 | 0.1 | — | — | — | — |
| Extract of Example 7 | — | 0.1 | — | — | — |
| Extract of Example 8 | — | — | 0.1 | — | — |
| Extract of Example 9 | — | — | — | 0.1 | — |
| Squalane | 5.0 | 5.0 | 5.0 | 5.0 | 5.0 |
| Beeswax | 4.0 | 4.0 | 4.0 | 4.0 | 4.0 |
| Polysorbate 60 | 1.5 | 1.5 | 1.5 | 1.5 | 1.5 |
| Sorbitan sesquioleate | 1.5 | 1.5 | 1.5 | 1.5 | 1.5 |
| Liquid paraffin | 0.5 | 0.5 | 0.5 | 0.5 | 0.5 |
| Caprylic/capric | 5.0 | 5.0 | 5.0 | 5.0 | 5.0 |
| triglyceride | | | | | |
| Glycerin | 3.0 | 3.0 | 3.0 | 3.0 | 3.0 |
| Butylene glycol | 3.0 | 3.0 | 3.0 | 3.0 | 3.0 |
| Propylene glycol | 3.0 | 3.0 | 3.0 | 3.0 | 3.0 |
| Carboxyvinyl polymer | 0.1 | 0.1 | 0.1 | 0.1 | 0.1 |
| Triethanolamine | 0.2 | 0.2 | 0.2 | 0.2 | 0.2 |
| Preservative, pigment, | q.s. | q.s. | q.s. | q.s. | q.s. |
| fragrance | | | | | |
| Purified water | Balance | Balance | Balance | Balance | Balance |

### TEST EXAMPLE 3

### Test for Skin Whitening Effect on Human Skin

An opaque tape having a perforation (1.5 cm (W)×1.5 cm (L)) was attached to the upper arm portion of each of 12 healthy men, and then the attached portion was radiated with UVB at a dose about 1.5-2 times the minimal erythema dose of each subject to induce skin darkening.

After the UV radiation, each of Formulation Examples 1 to 9 and Comparative Formulation Example 1 was applied on the UV-radiated portion, and a control portion (not applied with anything) was provided. After 6 weeks, the change in the state of the skin was observed. The color of the skin was measured using the colorimeter CR2002 (Japan, Minolta). The difference (ΔL*) in skin color between the time point of initiation of application and the time point of completion of application of each formulation was calculated according to the following equation 3, and the calculation results are shown in Table 5 below. Meanwhile, the whitening effect is evaluated by comparing the ΔL* value between the sample-applied portion and the control portion.

ΔL*=L* value at time point of measurement−L* value after induction of darkening at initial stage of measurement [Equation 3]

TABLE 5

| Test material | Lightness (ΔL*) of skin color |
|---|---|
| Formulation Example 1 | 1.5 |
| Formulation Example 2 | 1.4 |
| Formulation Example 3 | 1.2 |
| Formulation Example 4 | 1.0 |
| Formulation Example 5 | 1.1 |
| Formulation Example 6 | 1.3 |
| Formulation Example 7 | 0.9 |
| Formulation Example 8 | 1.5 |
| Formulation Example 9 | 1.3 |
| Comparative Formulation Example 1 | 0.6 |

As can be seen from the results in Table 5 above, the skin-whitening cosmetic composition according to the present invention exhibits a high skin-whitening effect.

Meanwhile, the formulation of the skin-whitening cosmetic composition according to the present invention can be suitably selected, and examples thereof include nourishing cream, milk lotion, massage cream, nourishing essence, skin lotion and the like.

In addition, components other than the paper mulberry extract in each cosmetic formulation can be suitably selected by those skilled in the art depending on the intended use of the cosmetic formulation.

### FORMULATION EXAMPLE 10

### Skin Lotion

A skin lotion of Formulation Example 10 was prepared using the composition shown in Table 6 below according to a conventional method (unit: wt %).

TABLE 6

| Component | Content |
|---|---|
| Extracts of Examples 1 to 9 | 10.0 |
| Glycerin | 3.0 |
| Butylene glycol | 2.0 |
| Propylene glycol | 2.0 |
| Carboxyvinyl polymer | 0.1 |
| PEG12 nonylphenylether | 0.2 |
| Polysorbate 80 | 0.4 |
| Ethanol | 10.0 |
| Triethanolamine | 0.1 |
| Preservative, pigment, fragrance | q.s. |
| Purified water | Balance |

### FORMULATION EXAMPLE 11

### Nourishing Cream

A nourishing cream of Formulation Example 11 was prepared using the composition shown in Table 7 below according to a conventional method (unit: wt %).

TABLE 7

| Component | Content |
|---|---|
| Extracts of Examples 1 to 9 | 10.0 |
| Polysorbate 60 | 1.5 |
| Sorbitan sesquioleate | 0.5 |
| Liquid paraffin | 2.0 |
| Squalane | 10.0 |
| Caprylic/capric triglyceride | 5.0 |
| Glycerin | 5.0 |
| Butylene glycol | 5.0 |
| Propylene glycol | 3.0 |
| Carboxyvinyl polymer | 3.0 |
| Triethanolamine | 0.2 |
| Preservative, pigment, fragrance | q.s. |
| Purified water | Balance |

### FORMULATION EXAMPLE 12

### Massage Cream

A massage cream of Formulation Example 12 was prepared using the composition shown in Table 8 below according to a conventional method (unit: wt %).

TABLE 8

| Component | Content |
|---|---|
| Extracts of Examples 1 to 9 | 10.0 |
| Beeswax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerin | 5.0 |
| Butylene glycol | 3.0 |
| Propylene glycol | 3.0 |
| Triethanolamine | 0.2 |
| Preservative, pigment, fragrance | q.s. |
| Purified water | Balance |

### FORMULATION EXAMPLE 13

### Pack

A pack of Formulation Example 13 was prepared using the composition shown in Table 9 below according to a conventional method (unit: wt %).

TABLE 9

| Component | Content |
|---|---|
| Extracts of Examples 1 to 9 | 10.0 |
| Polyvinyl alcohol | 13.0 |
| Sodium carboxymethylcellulose | 0.2 |
| Glycerol | 5.0 |
| Allantoin | 0.1 |
| Ethanol | 6.0 |
| PEG 12 nonylphenylether | 0.3 |
| Polysorbate 60 | 0.3 |
| Preservative, pigment, fragrance | q.s. |
| Purified water | Balance |

FORMULATION EXAMPLE 14

### Gel

A gel of Formulation Example 14 was prepared using the composition shown in Table 10 below according to a conventional method (unit: wt %).

TABLE 10

| Component | Content |
|---|---|
| Extracts of Examples 1 to 9 | 10.0 |
| Sodium ethylenediamine acetate | 0.05 |
| Glycerin | 5.0 |
| Carboxyvinyl polymer | 0.3 |
| Ethanol | 5.0 |
| PEG 60 hydrogenated castor oil | 0.5 |
| Triethanolamine | 0.3 |
| Preservative, pigment, fragrance | q.s. |
| Purified water | Balance |

1. A composition for skin whitening, comprising, as an active ingredient, a dried powder of an extract of a plant, said plant being one or more selected from the group consisting of Wikstroemia trichotoma and Edgeworthia chrysantha, wherein the extract is obtained by extracting the plant with water and then with an ethanol.

2. The composition of claim 1, wherein the dried powder is contained in an amount of 0.0001-90 wt % based on the total weight of the composition.

3. The composition of claim 1, wherein the composition is formulated as a cosmetic product, a medical drug or a food.

4. The composition of claim 1, wherein the ethanol extraction is carried out under reflux.

5. The composition of claim 4, wherein the ethanol is 70% ethanol.

6. A method for whitening skin, comprising topically applying the composition of claim 1 to the skin.

7. A method of claim 6, wherein the composition contains the dried powder in an amount of 0.0001-90 wt % based on the total weight of the composition.

8. The method of claim 6, wherein the ethanol extract is obtained by carrying out the ethanol extraction out under reflux.

9. The method of claim 8, wherein the ethanol is 70% ethanol.
